(19) United States
(12) Patent Application Publication
(10) Pub. No.: US 2010/0048728 A1
(43) Pub. Date: Feb. 25, 2010

(54) POLYCYCLIC DIANTHRAQUINONES AS INHIBITORS OF INFLAMMATORY CYTOKINES

(75) Inventors: Zadik Hazan, Zichron Yaakov (IL); Ruth Cohen, Neveh Ephraim (IL)

Correspondence Address: ROTHWELL, FIGG, ERNST & MANBECK, P.C., 1425 K STREET, N.W., SUITE 800, WASHINGTON, DC, 20005, US

(73) Assignee: HY BIOPHARMA INC., Jamison, PA (US)

(21) Appl. No.: 12/375,968

(22) PCT Filed: Aug. 2, 2007
(86) PCT No.: PCT/IL07/00967

§ 371 Date: Nov. 9, 2009

Related U.S. Application Data
(60) Provisional application No. 60/835,098, filed on Aug. 3, 2006.

(51) Int. Cl. A61K 31/122; A61P 29/00

(52) U.S. Cl. 514/680

(57) ABSTRACT

The present invention relates to the therapeutic use of polycyclic dianthraquinones including hypericin, helianthrones and helianthrone derivatives as inhibitors of the proinflammatory cytokine cascade mediated inflammatory conditions such as inflammatory bowel disease and cachexia. In addition the compositions can be used for prevention of development of inflammation, fibrosis and vasculopathy as a sequelae induced by irradiation treatment, and to the use thereof, as protective modes for the consequences of irradiation treatment in diseases such as cancers. The present invention can also be used to prevent or improve the inflammatory consequences of infections with viruses or bacteria and to prevent or treat neuroinflammatory disorders.

### FIELD OF THE INVENTION

The present invention relates to therapeutic uses of polycyclic dianthraquinones including hypericin, helianthrone and helianthrone derivatives as inhibitors of the inflammatory cytokine cascade, for treatment of cytokine mediated inflammatory conditions particularly inflammatory bowel disease and cachexia, for treatment of neuroinflammatory conditions, and for treatment and prevention of inflammation, fibrosis and vasculopathy as sequelae induced by irradiation treatment.

### BACKGROUND OF THE INVENTION

### Perylene Quinones

Perylene quinones are a unique group of red-ox reagents which have been found to exert a variety of biological effects which include immunomodulation and inhibition of a selected group of protein kinases. The first of these compounds to be thoroughly evaluated was hypericin, a potent photodynamic agent initially discovered to be virucidal to retroviruses (Lavie et al., 1989; Meruelo et al., 1988), and subsequently to all lipid-enveloped viruses (Tang et al., 1990). Additional studies identified hypericin as a potent and irreversible light-dependent inhibitor of protein kinase C (PKC), particularly when PKC is translocated to the cell membrane following cell activation (Takahashi et al., 1989).

Hypericin and related polycyclic aromatic compounds, generally of plant origin, are known to be useful in treating a variety of diseases that are caused by viruses and retroviruses (Lavie et al., 1989). U.S. Pat. No. 4,898,891 discloses the antiviral activity of two aromatic polycyclic dione compounds, hypericin and pseudohypericin and related compounds. U.S. Pat. No. 5,047,435 expands upon the disclosure of U.S. Pat. No. 4,898,891, and discloses the use of hypericin and pseudohypericin as effective anti-retroviral agents. U.S. Pat. No. 5,506,271 discloses methods of treating papilloma virus infection using hypericin. U.S. Pat. No. 5,149,718 discloses hypericin related compound containing compositions and methods for inactivating viruses and retroviruses present in bloods, blood products and other body fluids and, more generally biological fluids, as well as articles useful for practicing such methods. U.S. Pat. No. 6,150,414 discloses compositions comprising aromatic polycyclic antiviral compounds and methods for treating viral infections. These patents relate to prevention of viral infection and do not teach or suggest treatment of inflammation due to viral infection. U.S. Pat. Nos. 6,056,961 and 6,428,819 disclose plant extracts for the preparation of pharmaceutical compositions for the treatment of hepatitis.

WO 99/06347 discloses use of hypericin for treatment of tumors using photodynamic therapy (PDT) for eliciting destruction of tumors in conjunction with light irradiation. WO 01/56558 discloses use of hypericin for treatment of cancer in the absence of light irradiation by inhibiting transduction of signals for cell proliferation and cell progression through the cell replication cycle and use of hypericin for treating opthalmologic disorders associated with angiogenesis and prevention of formation of metastases.

U.S. Pat. No. 5,514,714, WO 93/08797 and WO 92/03049 disclose the use of polycyclic aromatic compounds, preferably hypericin or pseudohypericin for treating T cell-mediated diseases in mammals, when administered in alone or in combination with an immunosuppressive agent. According to these disclosures, the treatment inhibits the lytic phase of the cytotoxic effector T-lymphocytes via reduction in the expression of CD4 molecules on said cells. There is no teaching or suggestion of TNF inhibitory activity mediated by Perylene quinones.

### Radiation Therapy

Radiotherapy is a very significant treatment modality for many forms of Cancer. Given the incidence of cancer in the population and the prevailing assessment that more than 50% of cancer patients benefit from inclusion of radiotherapy in their treatment, more than 10% of the population are likely to experience cancer radiotherapy in their lifetime. The effective dose in most cases of cancer radiotherapy is damaging to the normal tissues.

Many different types of compounds have been proposed as useful for preventing or ameliorating radiation damage including fibrotic damage resulting from radiation therapy of cancers. Nevertheless, no satisfactory therapy exists at present to prevent secondary radiation damage.

Lung cancer is relatively resistant to radiation and radiation pneumonitis is a major obstacle to increasing the radiation dose. Exposure of the lungs to ionizing radiation induces pneumonitis as an acute phase which subsides after a few weeks, and followed by inflammation and fibrosis as the chronic phase that can develop months or years after irradiation (Stone et al. 2003).

In addition, in most cases the normal tissues surrounding the specific site of irradiation are also affected and damaged. Although there are advances in understanding the cellular and molecular biology of radiation fibrosis this chronic phase is refractory to treatment, including steroids treatment (Abratt R P et al. 2004). Therefore it is important to prevent those reactions shortly after radiation itself in order to minimize the consequences of the release of proinflammatory cytokines.

Recently, advances in understanding of cellular and molecular biology have provided new insights into mechanisms of radiation fibrosis. In the treatment of acute pneumonitis, high dose steroid therapy and other types of therapy are useful and essential but late radiation lung fibrosis is refractory to treatment; therefore, the particularly important point is to minimize the likelihood of development of fibrosis (Abratt et al. 2004). Lately, (Chen et al. 2005) caffeic acid phenethyl ester was described as an agent capable of decreasing acute pneumonitis after irradiation of normal lung tissue in vitro and in vivo.

Irradiation of other organs and tissues within the human body also results in inflammatory reaction and damage to the normal tissue surrounding the tumor. In radical therapy of pelvic cancer, during five or six week course of treatment, approximately 80% of patients develop gastrointestinal symptoms which are partly caused by acute gastrointestinal inflammation. (Andreyev J, 2005). A well recognized complication of pelvic field radiation therapy is proctitis, with a frequency between 5% and 20% (Buchi 1991). Manifestations include tenesmus, bleeding, diarrhea, and fecal incontinence (Moore et al. 2000). The proctitis is mostly due to the vasculopathy which may appear as late as one year or more after the exposure to pelvic irradiation. Irradiation of brain tumors is associated with long term complications of radiation therapy such as encephalopathy and growth and development impairment and vasculopathy of the arteries. Injury to the nervous system is a common complication of radiation therapy. In adults, the incidence of radiation necrosis after conventional doses for brain tumor therapy ranges from 5 to 24%. However, a non-nercotizing form of encephalopathy with neurobehavioral manifestations is even more common and is seen even with lower dosages (Perry and Schmidt 2006).

Zhang et al. 1996 suggest a role for hypericin as a radiosensitizor for malignant glioma. The article describes enhancement in radiosensitivity of human malignant glioma cells by hypericin in vitro, when administered prior to the radiotherapy. The authors speculate that the activity of hypericin is due to its protein kinase C (PKC) inhibitory activity. No other cell lines were tested for the activity of hypericin together with radiation, nor treatment of cells with hypericin together or following exposure to radiation was tested.

U.S. Pat. No. 6,630,473 discloses anti-inflammatory agents and inhibitors useful against increase in ocular tension caused by irradiation with lasers. U.S. Pat. No. 6,194,414 discloses a method of cancer radiotherapy and a method of protecting a subject from radiation damage using the specific radioprotector denoted ortho methyl para dimethylamino Hoechst.

### Tumor Necrosis Factor Induced Proinflammatory Cytokine Cascade:

Tumor necrosis factor α (TNFα) is recognized as being involved in the pathology of many infectious and auto-immune diseases (W. Fiers, 1991). Furthermore, it has been shown that TNF is the prime mediator of the inflammatory response seen in sepsis and septic shock (C. E. Spooner et al., Clinical Immunology and Immunopathology, 1992, 62, p. S11), an often fatal condition associated with Gram-negative or Gram-positive bacteremia, as well as in other conditions such as adult respiratory distress syndrome and graft-versus-host disease. TNF is also a key mediator in a number of autoimmune and inflammatory diseases such as rheumatoid arthritis, cerebral malaria and multiple sclerosis (reviewed in Tracy, K. J. and Cerami, 1993). Antagonistic TNF treatment with anti-TNF antibodies and dimeric TNF-receptor-IgG fusion chimeras have shown promising therapeutic results for a variety of diseases in animal models and human clinical trials (Elliot, M., et al. 1993) and are currently used for treatment of rheumatoid arthritis (Chen et al. 2006).

It is well known that radiation causes local and systemic cytokine production which may begin immediately after the exposure and can persist for several weeks or months (Tartakovsky et al. 1993, Morgan and Breit 1995, Rubin et al. 1995). The lung damage which is caused by radiation may be attributed to the rise in the synthesis of pro-inflammatory cytokines such as interleukin 1β (IL-1β), interleukin 6(IL-6), TNFα, fibroblasts growth factor like TGF β, and vascular adhesion molecules such as ICAM-1, ELAM-1 and selectins (Hallahan et al. 1989, Krivenko et al. 1992, Johnston et al. 1996, Thornton et al. 1996, Hallahan & Virudachalam 1997b). Several studies have shown that TNFα is constitutively expressed in lung epithelial cells, which leads in turn to progressive mononuclear infiltration and lung fibrosis (Miyazaki et al. 1995, Thrall et al. 1997, Sime et al. 1998). Circulating cytokines produced by cells outside the lung as a consequence of total body irradiation also contribute to the development of complications in the lung (Shankar and Cohen, 2001, Hong et al. 1999).

### Inflammatory Bowel Disease

Inflammatory bowel disease (IBD) refers to chronic disorders (primarily Crohn's disease and ulcerative colitis) that cause inflammation or ulceration in the small and large intestines. Although there are many documented patterns of prevalence, it is a disease of unknown cause. IBD has no medical cure, and once the diseases are manifest, they tend to fluctuate between periods of remission and relapse. The development of IBD is likely multi-factorial, but appears to involve a dysregulated immune response to pathogenic and/or resident normal bacteria in a genetically pre-disposed host. Due to the significant role of bacterial infection in IBD and related conditions, one therapeutic strategy relies upon use of antibiotics. IBD affect approximately 500,000 to 2 million people in the United States.

IBD is often characterized with alternating periods of remission followed by periods of unpredictable relapse or flare-up of varying severity. About 50% of patients are in remission at any given time and the majority suffers at least one relapse in a 10-year period. In addition, there are many systemic complications that accompany this disease with the most common being arthritis. Symptoms of arthritis occur in one fourth of all people with IBD. Joint inflammation occurs most often when the colon is involved in the disease process and flares when the bowel disease is most active. This form of inflammatory arthritis does not cause permanent deformity and is often short lived.

At present, there is no cure for IBD. Many of the current therapeutic agents focus on controlling the disease symptoms by suppressing the inflammation associated with the disease. The principal drugs used to treat IBD are aminosalicylates and corticosteroids and for those individuals that do not respond well to these agents, antibiotics and immunosuppressive medications can also be used. A recently introduced humanized anti TNFα antibody (Infliximab) has been found to provide considerable relief to patients from disease symptoms, however serious toxicities related to the therapies have emerged and its safety profile is in doubt (Carroccio et al 2006). Although drug treatment is effective for 70 to 80% of patients, surgery is often required for individuals having more active disease.

### Inflammatory Sequelae of Infections by Foreign Invaders Such as Bacteria or Viruses

Immuno-inflammatory reactions occur in response to several viruses including influenza, respiratory syncytial virus infection, herpes infection and varicella zoster (shingles), The inflammatory responses to varicella result in severe and painful episodes which are generalized and affect the entire back and chest or other organs, last for between one month to several months and bring about complete incapacitation of affected individuals.

Herpes simplex virus (HSV) causes productive and latent forms of infection in humans and experimental animals. The primary infection and reactivation of the latent infection evoke an immune response in the host organism, involving activities of macrophages, CD4+ and CD8+ T lymphocytes, and B lymphocytes. Strong cytokine responses are associated with the acute and recurrent phases of HSV infection. During the latent phase of HSV infection in the sensory ganglia, expression of certain cytokines can be detected. The cytokine response to HSV infection is dominated by proinflammatory and Th1 type cytokines (Hukkanen V. 2002, Int. Rev. of Immunol. 21; 355-371. Macrophage activation has also been associated with proinflammatory cytokine release such as TNFα (Paludan et al. 2001).

Acne and Rosacea are common skin diseases which involve inflammation. In many cases inflammation in acne is wide spread and deforming leading it to be classified as inflammatory acne. The pathogenesis of acne is multifactorial including hormonal, microbiological and immunological mechanisms (Kim J. 2002).

One of the contributors to the pathogenesis of acne is Propionibacterium acnes. P. acnes contributes to the inflammatory nature of acne by inducing monocytes to secrete TNFα, IL-1β, and IL-8 (Vowels 2004).

### Neurodegeneration

TNFα level is upregulated and contributes to the pathogenesis of neurodegenerative diseases, such as Alzheimer's disease, multiple sclerosis, Parkinson's disease and the degeneration of the optic nerve in glaucoma (Shoami et al. 1999). TNF-α is activating the glial cells which in turn secrete cytotoxic cytokines which lead to neuron and oligodendrocyte death (Nakazawa et al. 2006).

Thus there is a widely recognized unmet need for compounds useful for the inhibition and prevention of the proinflammatory cytokine cascade, induced by TNFα in neurodegenerative disorders and inflammation mediated pathological conditions which for example follow radiation therapy protocols of cancers and other disorders.

### SUMMARY OF THE INVENTION

The present invention provides therapeutic uses of polycyclic dianthraquinones including hypericins and helianthrones as inhibitors of the TNFα proinflammatory cytokine cascade. The present invention provides therapeutic uses of polycyclic dianthraquinones including hypericins and helianthrones as inhibitors of the proinflammatory cytokine cascade for inhibiting, preventing or ameliorating the development of conditions associated with inflammation, and as inhibitors of the development of inflammation, fibrosis and vasculopathy as late chronic sequelae to irradiation treatment, or in response to viral or bacterial infections.

The present invention further provides methods of use of hypericin and related polycyclic aromatic compounds, for preventing cytokine induced damage to normal tissues resulting from exposure to radiation. More specifically, the present invention provides methods of use of hypericin and related polycyclic aromatic compounds as protective agents for preventing development of inflammation included fibrosis, vasculopathy and other sequelae, following ionizing irradiation treatment (of cancers and other disorders), for protecting a subject from radiation damage.

It is now disclosed for the first time that a therapeutically effective amount of pharmaceutical compositions comprising hypericin, helianthrone and their derivatives, administered to the subject after exposure to irradiation, can protect biological material of the subject from late and chronic inflammatory reactions caused by radiation. It is also shown for the first time that hypericin, helianthrone and their derivatives are effective in inhibiting the proinflammatory cytokine cascade and can be therefore used for treating inflammatory bowel disease and other diseases associated with TNFα activity. Furthermore, these compounds are active in protection and rescue of neural cells from death induced by TNFα and therefore are a valuable treatment in neurodegenerative disorders and glaucoma. The protection of TNFα induced toxicity to neural cells by hypericin is highly potent and can prevent TNFα induced neural cell death in neurodegenerative and neuroinflammatory disorders.

The present invention is based in part on the surprising finding that hypericin is capable of preventing or inhibiting the TNFα induced proinflammatory cytokine cascade and inflammation, fibrosis and vasculopathy associated with irradiation treatment.

It is now disclosed that the compositions of the present invention comprising hypericin and helianthrone derivatives are unexpectedly effective, when administered together with or even after exposure to radiation, in reducing or preventing undesired late reaction to irradiation treatment in normal tissue or cells.

The present invention thus provides, in one aspect, use of a compound of general formulae (Ia) and (Ib):

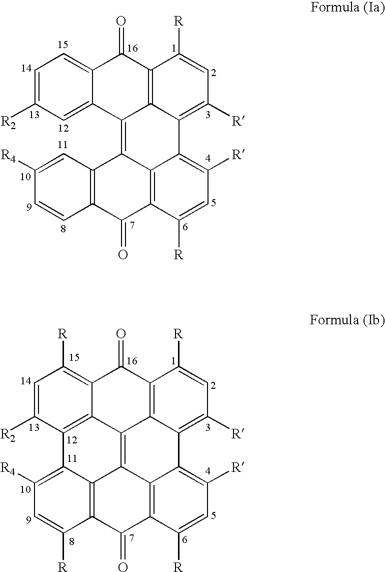

wherein R is selected from the group consisting of hydroxy, C₁-C₁₀ alkoxy, NH—C₁-C₁₀ alkyl, and NH-hydroxy(C₁-C₁₀)alkyl; R′ is selected from the group consisting of hydroxy and C₁-C₁₀ alkoxy; and R₂ and R₄ are independently selected from the group consisting of hydrogen, hydroxy, chloro, bromo, C₁-C₁₀ alkyl, C₁-C₁₀ alkoxy, and C₁-C₁₀ alkoxycarbonyl; for inhibition of TNF mediated inflammatory cytokine cascade, for treatment of cytokine mediated inflammatory conditions and for prevention, amelioration or treatment of inflammation, fibrosis and vasculopathy caused by irradiation and other conditions such as viral or bacterial infections.

The helianthrone derivatives are those presented by Formula (Ia), wherein there is no bond between positions 11 and 12 and there is H at positions 8 and 15. The hypericin derivatives are those presented by Formula (Ib) wherein there is an additional ring formed by the bond between positions 11 and 12 and R is other than H at positions 8 and 15.

Examples of such compounds as the currently most preferred embodiments of the present invention are hypericin, 10,13-dimethyl-1,3,4,6-tetrahydroxyhelianthrone and 10,13-dimethyl-1,3,4,6-tetramethoxyhelianthrone of formulas A, B and C below:

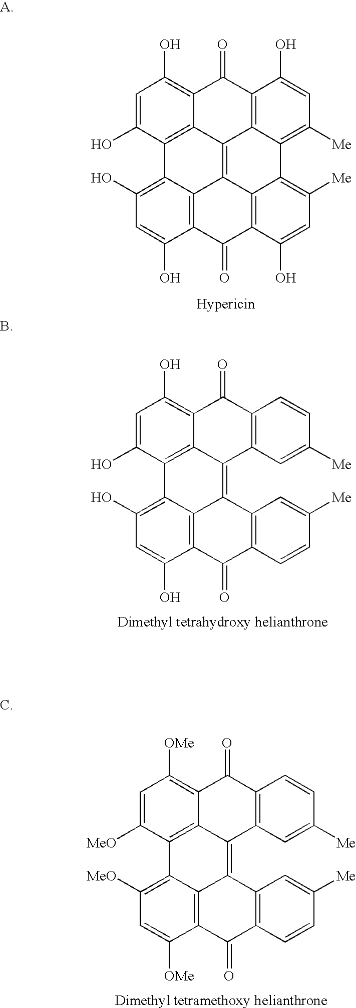

Additional embodiments include, but are not limited to of helianthrone derivatives selected from the group consisting of:
1,3,4,6-tetrahydroxyhelianthrone;
1,3,4,6-tetramethoxyhelianthrone;
1,6-di-N-butylamino-3,4-dimethoxy-helianthrone;
1,6-di-N-butylamino-3,4-dimethoxy-10,13-dimethyl-helianthrone;
1,6-di-(N-hydroxyethylamino)-3,4-dimethoxy-helianthrone;

According to one embodiment, the present invention provides a pharmaceutical composition comprising a compound of general formulae (Ia):

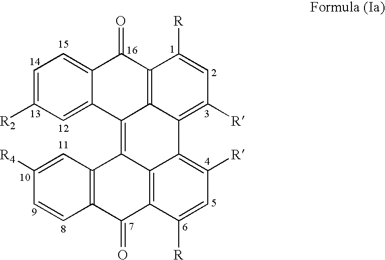

wherein R is selected from the group consisting of hydroxy, C₁-C₁₀ alkoxy, NH—C₁-C₁₀ alkyl, and NH-hydroxy(C₁-C₁₀)alkyl; R′ is selected from the group consisting of hydroxy and C₁-C₁₀ alkoxy; and R₂ and R₄ are independently selected from the group consisting of hydrogen, hydroxy, chloro, bromo, C₁-C₁₀ alkyl, C₁-C₁₀ alkoxy, and C₁-C₁₀ alkoxycarbonyl; for inhibition of TNF mediated inflammatory cytokine cascade, for treatment of cytokine mediated inflammatory conditions and for prevention, amelioration or treatment of inflammation, fibrosis and vasculopathy caused by irradiation and other conditions such as viral or bacterial infections.

According to another embodiment, the present invention provides a pharmaceutical composition comprising a compound of general formulae (Ib):

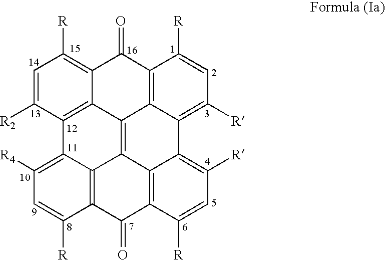

wherein R is selected from the group consisting of hydroxy, C₁-C₁₀ alkoxy, NH—C₁-C₁₀ alkyl, and NH-hydroxy(C₁-C₁₀)alkyl; R′ is selected from the group consisting of hydroxy and C₁-C₁₀ alkoxy; and R₂ and R₄ are independently selected from the group consisting of hydrogen, hydroxy, chloro, bromo, C₁-C₁₀ alkyl, C₁-C₁₀ alkoxy, and C₁-C₁₀ alkoxycarbonyl; for inhibition of TNF mediated inflammatory cytokine cascade, for treatment of cytokine mediated inflammatory conditions and for prevention, amelioration or treatment of inflammation, fibrosis and vasculopathy caused by irradiation and other conditions such as viral or bacterial infections.

According to one embodiment, the pharmaceutical composition comprising a compound of the general formula (Ia) or (Ib) is for inhibiting of proinflammatory cytokine cascade and for treatment of disorders mediated by a proinflammatory cytokine cascade. According to one embodiment the proinflammatory cytokine is selected from the group consisting of TNFα, interleukin (IL)-1beta, IL-6, IL-18 or HMG-1 (high mobility group 1). According to a specific embodiment the proinflammatory cytokine is TNFα.

In another aspect, the present invention provides pharmaceutical compositions comprising a compound of the general Formula (Ia) or Formula (Ib) as described above for the uses as described above.

In yet another aspect, the present invention provides a method for the inhibition of proinflammatory cytokine cascade, for treatment of cytokine mediated inflammatory conditions which arise in response to infection with a virus or a bacteria, and for prevention, amelioration or treatment of inflammation, fibrosis and vasculopathy caused by irradiation which comprises administering to a patient in need thereof a pharmaceutical composition comprising an effective amount of a compound of the general Formula (Ia) or Formula (Ib). The method comprises treating the patient with a pharmaceutical composition comprising a compound of the general Formula (Ia) or Formula (Ib) in an amount sufficient to inhibit the inflammatory cytokine cascade, wherein the patient is suffering from, or at risk for, a condition mediated by the inflammatory cytokine cascade.

According to the present invention, any condition, mediated by TNFα is potential for being treated with a pharmaceutical composition comprising at least one compound of the general Formula (Ia) or Formula (Ib). According to one embodiment, the condition mediated by the TNF mediated inflammatory cytokine cascade, which may be treated, is selected from the group consisting of: inflammatory bowel disease, ulcerative, acute or ischemic colitis, Crohn's disease and cachexia (wasting syndrome). According to another embodiment, the condition involves a bacterial infection. According to a specific embodiment the condition is septic shock (sepsis, endotoxic shock) or disseminated bacteremia. According to yet another embodiment, the condition is a neurodegenerative disorder. According to a specific embodiment the neurological disorder is selected from the group consisting of Alzheimer's disease (AD), neurological lesions associated with diabetic neuropathy, demyelinating disorders other than autoimmune demyelinating disorders, retinal degeneration, muscular and glaucoma. According to a specific embodiment the TNFα mediated condition to be treated according to the invention is glaucoma, in which the compounds administered inhibit the TNFα mediated neural injury.

According to a another embodiment, the present invention provides a method for treating sequelae of radiotherapy comprising administering to a patient in need thereof a pharmaceutical composition comprising an effective amount of a helianthrone compound of the Formula (Ia) or Formula (Ib).

Non-limiting examples of irradiation types which may be treated according to the method of the present invention are: ionizing radiation, laser irradiation, microwave radiation, ultraviolet, infrared radiation and ultrasonic thermotherapy. The conditions treated with radiation, in which the compositions of the present invention may be applied include but are not limited to cancers, and intraocular inflammation (caused for example by laser irradiation). According to a specific embodiment the radiation is ionizing radiation applied in cancer radiotherapy.

According to a specific embodiment, a method for prevention and treatment of inflammation, fibrosis and vasculopathy as sequelae following irradiation treatment of cancers, comprising administering a pharmaceutical composition comprising a compound of the general Formula (Ia) or Formula (Ib) is provided. The cancers include but not being limited to, lung cancer, breast cancer, prostate cancer, brain cancer and all forms of cancer in which radiation is required.

According to one embodiment, the pharmaceutical composition comprising a compound of the general Formula (Ia) or Formula (Ib) is administered after exposure to any type of irradiation and prevents or reduces the late inflammatory reaction caused to normal tissue by the irradiation. According to a specific embodiment the pharmaceutical composition is administered following the completion of the irradiation treatment.

According to another embodiment, the pharmaceutical composition comprising a compound of the general Formula (Ia) or Formula (Ib) is administered to the subject in need thereof together with the exposure to radiation.

In another embodiment, the pharmaceutical composition comprising a compound of the general Formula (Ia) or Formula (Ib) is for prevention and treatment of local or generalized inflammation condition initiated by infection with viruses or bacteria. While such compounds are known to prevent or reduce viral infection, it is now shown for the first time that these compounds are also capable of inhibiting inflammation caused by viral or bacterial infection.

According to a specific embodiment the viral infection is selected from the group consisting of: influenza, respiratory syncytial virus infection, herpes infection and varicella zoster (shingles). According to another specific embodiment the bacteria is Propionibacterium acnes and the compound of the general Formula (Ia) or Formula (Ib) is used for treatment of Acne or Rosacea.

According to another embodiment of the present invention, the medicament comprising a compound of the general Formula (Ia) or Formula (Ib) is administered to the subject in need thereof following development of a fulminant infection with herpes virus or with the varicella zoster virus (which causes shingles) or with the chicken pox virus.

The choice of the pharmaceutical additives, carriers, diluents, excipients and the like, will be determined in part by the particular active ingredient, as well as by the particular route of administration of the composition. The routes of administration include but are not limited to oral, aerosol, parenteral, topical, ocular, transdermal, subcutaneous, intravenous, intramuscular, intraperitoneal, intrathecal, rectal and vaginal systemic administration. In addition, the pharmaceutical compositions of the invention can be directly delivered into the central nervous system (CNS) by intracerebroventricular, intraparenchymal, intraspinal, intracisternal or intracranial administration.

The pharmaceutical compositions can be in a liquid, aerosol or solid dosage form, and can be formulated into any suitable formulation including, but not limited to, solutions, suspensions, micelles, emulsions, microemulsions, aerosols, powders, granules, sachets, soft gels, capsules, tablets, pills, caplets, suppositories, creams, gels, pastes, foams and the like, as will be required by the particular route of administration.

These and additional benefits and features of the invention will be better understood with reference to the following detailed description taken in conjunction with the figures and non-limiting examples.

### BRIEF DESCRIPTION OF THE DRAWINGS

FIG. 1 describes experimental results showing that Hypericin administration prevents colon shortening in mice with IBD.

FIG. 2 illustrates the protective effect of Hypericin from TNF induced cell death of L-cells, analyzed by MTT assay.

FIG. 3 depicts the protective effect of Hypericin from TNF induced cell death of L-cells analyzed by Hemacolor assay.

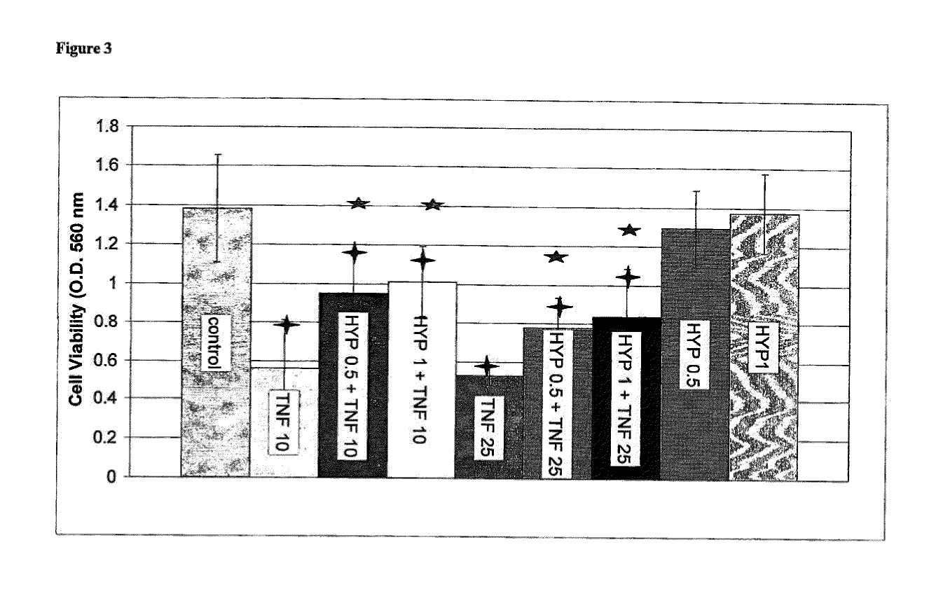

FIG. 4 presents results of experiments showing that Hypericin administration prevents the inflammation-related erythema, vasodilation and edema caused by inoculation of herpes virus to the backs of guinea pigs.

### DETAILED DESCRIPTION OF THE INVENTION

The present invention is based on the unexpected finding that hypericin and related compounds are useful for preventing cytokine induced fibrosis and vasculopathy leading to normal tissue damage resulting from exposure to ionizing radiation. More specifically, the present invention provides methods of use of hypericin and related polycyclic aromatic compounds as protective agents to prevent development of inflammation fibrosis, vasculopathy and other sequelae following irradiation treatment of cancers and other disorders for protecting a subject from radiation damage by administering to the subject, after exposure to irradiation, a therapeutically effective amount of pharmaceutical compositions comprising hypericin, helianthrone and their derivatives to protect biological material of the subject from late chronic inflammatory reaction.

Thus it is now disclosed that hypericin and helianthrone as well as their derivatives are useful in the inhibition of the inflammatory cytokine cascades resulting from exposure of cells to irradiation.

The present invention relates to use of hypericin, helianthrone and their derivatives in prevention of cytokine mediated, post radiation late consequences such as fibrosis, vasculitis, proctitis and apoptosis of neurons in the brain which causes cognitive impairment.

The dominant consideration in determining radiation doses for cancer radiotherapy is the assessment of tolerance of the most radiosensitive normal tissues/organs in the treatment field. The problem is that late adverse reactions develop at various time points following termination of irradiation in the adjacent tissues. These adverse effects are the result of some tissue damage caused by radiation to the adjacent tissues. This damage is associated with release of inflammation-inducing cytokines which progressively exacerbate the initial damage. The treatment of tumors with ionizing radiation (hereinafter referred to as “cancer radiotherapy”) is used extensively in cancer therapy. The goal of such treatment is to elicit the destruction of tumor cells and inhibition of tumor cell growth presumably through DNA damage, while causing minimum damage to non-tumor cells and tissues. Collateral damage to adjacent tissues often limits the effectiveness of radiotherapy of certain tumors, such as brain tumors and tumors in the abdominal cavity and the neck.

The radiation damage may result from exposure to a radiation source, such as, ionizing radiation. The term “ionizing radiation” as used herein refers to photons having enough energy to ionize a bond, such as, α, β and γ rays from radioactive nuclei and x-rays. Other types of radiation which may damage the normal tissue are laser irradiation, microwave irradiation, ultraviolet radiation, infrared radiation or ultrasonic thermotherapy. The applied radiation may be used locally, to a specific organ or tissue or it may be a total body radiation.

In another aspect, the present invention provides a method for inhibition or reduction of inflammatory cytokine cascade sequelae and for a treatment of cytokine mediated inflammatory conditions which arise in response to infection with a microbial agent such as a virus or a bacteria. Immuno-inflammatory reactions occur in response to several viruses including influenza, respiratory syncytial virus infection, herpes infection and particularly varicella zoster (Shingles), chicken pox and other viruses.

TNFα activates glial cells which in turn secrete cytotoxic cytokines which lead to neuron and oligodendrocyte death and is therefore contributing to the pathogenesis of neurodegenerative diseases, such as Alzheimer's disease, and the degeneration of the optic nerve in glaucoma by. In the present invention it is shown that hypericin, and related compounds prevents normal cell death induced by TNFα, and therefore is expected to be a valuable treatment in neurodegenerative disorders and in glaucoma.

One application of compositions of the present invention is after cancer radiotherapy. Many of the normal tissues which are adversely affected by radiotherapy such as the skin, oral mucosa, oesophageal mucosa, rectal mucosa, vaginal mucosa and bladder epithelium can be protected from the late effects which develop post irradiation. Thus, the application of compositions of the present invention could enable the use of higher radiation doses, as it offers a method to manage more severe post irradiation complications and can effectively limit their magnitude. The pharmaceutical composition may be administered to the subject in need thereof immediately after the last radiation session or up to two weeks following last irradiation treatment session.

Furthermore, the application of compositions of the present invention could also reduce the duration, severity and morbidity of inflammatory outcomes of infections with viral or bacterial pathogens, providing remedy to infections associated with major suffering.

The pharmaco-distribution properties of the compositions of the present invention offer other potential ways of achieving systemic protection of post-irradiation late complications. Examples include tumors in the brain and lung.

The compositions according to the present invention are unexpectedly effective anti-inflammatory agents useful to prevent or minimize the chronic inflammation related reaction of normal tissue following irradiation. Thus these compositions may be used in a variety of conditions and diseases involving treatment following radiation including but not limited to cancers, eye surgery and space occupying lesions. The compositions according to the present invention are further unexpectedly effective anti-inflammatory agents useful to prevent or minimize the chronic inflammation related reaction of normal tissue which occur during infections with viral or bacterial pathogens, and to prevent proinflammatory-induced neural cell death.

Inflammatory cytokine cascades contribute to deleterious outcomes, including inflammation and apoptosis, in numerous disorders. Included are disorders characterized by both localized and systemic reactions, including, without limitation, diseases involving the gastrointestinal tract and associated tissues (such as appendicitis, peptic, gastric and duodenal ulcers, peritonitis, pancreatitis, ulcerative, pseudomembranous, acute and ischemic colitis, diverticulitis, epiglottitis, hepatitis, Crohn's disease, and enteritis); systemic or local inflammatory diseases and conditions (such as asthma, allergy, anaphylactic shockhay fever, sepsis, septicemia, endotoxic shock, cachexia, and hyperpyrexia); diseases involving the urogential system and associated tissues (such as septic abortion, epididymitis, vaginitis, prostatitis and urethritis); diseases involving the respiratory system and associated tissues (such as bronchitis, rhinitis, adult respiratory distress syndrome, pneumonitis, alveolitis, bronchiolitis, pharyngitis, pleurisy, and sinusitis); diseases arising as immuno-inflammatory reactions occurring in response to infection by various viruses (such as influenza, respiratory syncytial virus, HIV, hepatitis B virus and herpes), bacteria (such as disseminated bacteremia, Dengue fever), inflammatory acne, and fungi (such as candidiasis); dermatological diseases and conditions of the skin (such as burns, dermatitis, urticaria, warts, and wheals); diseases involving the cardiovascular system and associated tissues (such as atherosclerosis, pericarditis, myocarditis, and rheumatic fever); diseases involving the central or peripheral nervous system and associated tissues (such as Alzheimer's disease, meningitis, encephalitis, neuralgia, spinal cord injury, and uveitis); diseases of the bones, joints, muscles and connective tissues (such as the various arthritides and arthralgias, osteomyelitis, fasciitis, periodontal disease, and synovitis); other inflammatory disorders (such as Type II diabetes, and ankylosing spondylitis); as well as various cancers, tumors and proliferative disorders (such as Hodgkins disease); and, in any case the inflammatory or immune host response to any primary disease. In all of these pathological conditions there is an inflammatory component which at times can be of significant severity and contribute to the disease morbidity. The compositions of the present invention can be used alone or in conjunction with other drugs to treat the non autoimmune diseases listed above.

As used herein “TNFα mediated condition” is intended to include a medical condition, such as a chronic or acute disease or pathology, or other undesirable physical state, in which a signaling cascade including TNFα plays a role, whether, for example, in development, progression or maintenance of the condition. Examples of TNFα mediated conditions include, but are not limited to: (A) acute and chronic immune, such as scleroderma and the like; (B) infections, including sepsis syndrome, circulatory collapse and shock resulting from acute or chronic bacterial infection, acute and chronic parasitic infection, and/or infectious diseases, whether bacterial, viral or fungal in origin, such as a HIV or AIDS, and including symptoms of cachexia, autoimmune disorders, Acquired Immune Deficiency Syndrome, dementia complex and infections; (C) inflammatory diseases, such as chronic inflammatory pathologies, including sarcoidosis, chronic inflammatory bowel disease, ulcerative colitis and Crohn's pathology, and vascular inflammatory pathologies, such as, disseminated intravascular coagulation, and Kawasaki's pathology; (D) neurodegenerative diseases, including, demyelinating diseases, such as acute transverse myelitis; and lesions of the corticospinal system; and mitochondrial multisystem disorder; demyelinating core disorders, such as acute transverse myelitis; and Alzheimer's disease; (E) malignant pathologies involving TNF-.alpha. secreting tumors or other malignancies involving TNF, such as leukemias including acute, chronic myelocytic, chronic lymphocytic and/or myelodyspastic syndrome; lymphomas including Hodgkin's and non-Hodgkin's lymphomas; and malignant lymphomas, such as Burkitt's lymphoma or Mycosis fungoides; and (F) alcohol-induced hepatitis See, e.g., Berkow, et al., eds., The Merck Manual, 16.sup.th edition, chapter 11, pp 1380-1529, Merck and Co., Rahway, N.J., (1992).

As used herein, the term “treating” means remedial treatment, and encompasses the terms “reducing”, “suppressing”, “ameliorating” and “inhibiting”, which have their commonly understood meaning of lessening or decreasing.

The term “post radiotherapy adverse effects in cancer” is used herein in its broadest sense and includes progressive post radiotherapy cytokine mediated damage to tissues adjacent to tumors which may be either benign or malignant and which were subjected to treatment with radiotherapy. All types of cancers, which may benefit from radiotherapy, are included within the list of possible diseases that are amenable to treatment to minimize radiation damage by using the compositions of the present invention.

The term sequelae of virus infection refers to the reddening (erythema), swelling (edema) and inflammatory vesicles which arise in different organs including but not limited to the skin (such as those which occur in chicken pox virus and herpes virus infections) and along nerve endings (such as in varicella zoster which are associated with severe pain).

The term sequelae of bacterial infection refers to the reddening (erythema) such as that which results from but not limited to Streptococcal infections in scarlet fever, edema and inflammation related tissue damage that leads to multiple organ failure during septic shock syndrome.

Compositions of the invention will be useful when the nerve damage results from neurodegenerative disorders, including, but not limited to, Alzheimer's disease (AD), neurological lesions associated with diabetic neuropathy, demyelinating disorders other than autoimmune demyelinating disorders, retinal degeneration, and peripheral neuropathies.

The term “wasting syndrome” is used herein to refer to cachexia associated profound weight loss and progressive depletion of muscle mass as is a common sequela of chronic diseases such as cancer, tuberculosis, bacterial septic shock and human immunodeficiency virus (HIV) infection. Unexpectedly, the compositions of the invention are now disclosed to be effective for the treatment of such disorders.

In the compounds of Formula (Ia) or Formula (Ib) used in the present invention, R is selected from the group consisting of hydroxy, C₁-C₁₀ alkoxy, NH—C₁-C₁₀ alkyl, and NH-hydroxy(C₁-C₁₀)alkyl; R′ is selected from the group consisting of hydroxy and C₁-C₁₀ alkoxy; and R₂ and R₄ are independently selected from the group consisting of hydrogen, hydroxy, chloro, bromo, C₁-C₁₀ alkyl, C₁-C₁₀ alkoxy, and C₁-C₁₀ alkoxycarbonyl.

As used herein, “C₁-C₁₀ alkyl, “C₁-C₁₀ alkoxy” and “C₁-C₁₀ alkoxycarbonyl” refer to straight or branched radicals having 1 to 10 carbon atoms. Examples of such alkyl radicals are, without being limited to, methyl, ethyl, propyl, isopropyl, butyl, hexyl, and octyl Examples of such alkoxy radicals are, without being limited to, methoxy, ethoxy, propyloxy, isopropyloxy, butoxy, hexyloxy, and octyloxy. Examples of such alkoxycarbonyl radicals are, without being limited to, methoxycarbonyl, ethoxycarbonyl, propyloxycarbonyl. In one preferred embodiment, R, R′ are methyl, but longer aliphatic chains envisaged in these positions instead of the methyl group may have advantages such as prolongation of biological activity due to better retention by cells and requiring less frequent administration.

The invention is exemplified by the compounds hypericin, helianthrone and derivatives thereof of Formula (Ia) and Formula (Ib) respectively wherein the two Rs at positions 1 and 6 are hydroxy, methoxy, butylamino or hydroxyethylamino, and the two R's at positions 3 and 4 are hydroxy or methoxycarbonyl. Examples of such preferred compounds are 1,3,4,6-tetrahydroxyhelianthrone, 1,3,4,6-tetramethoxyhelianthrone, 10,13-dimethyl-1,3,4,6-tetrahydroxyheliantlirone, 10,13-di(methoxycarbonyl)-1,3,4,6-tetramethoxyhelianthrone, 1,6-di-N-butylamino-3,4-dimethoxyhelianthrone, 1,6-di-N-butylamino-3,4-dimethoxy-10,13-dimethylhelianthrone, 1,6-di-(N-hydroxyethylamino)-3,4-dimethoxy-helianthrone, 2,5-dibromo-1,3,4,6-tetrahydroxyhelianthrone, and, most preferably, 10,13-dimethyl-1,3,4,6-tetramethoxyhelianthrone.

The compounds of the Formula (Ia) or Formula (Ib) according to the invention in which R₂ and R₄ are each lower alkyl can be prepared by the method described in U.S. Pat. No. 5,120,412 using as a starting material a 1,3-dihydroxy-6-(lower alkyl)-anthraquinone of the formula (II):

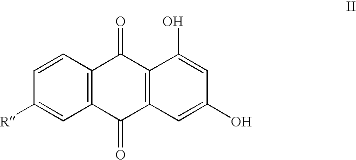

in which R′ is lower alkyl. Compound II is reduced to the corresponding anthrone of the formula (III)

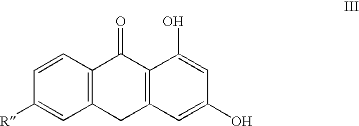

in which R′ is as defined above and compound III is condensed to obtain desired compounds of formula (I) in which R is lower alkoxy.

Other compounds of Formula (Ia) or Formula (Ib) can be prepared in an analogous manner using appropriately substituted 1,3-dihydroxy-anthraquinones.

The compounds of Formula (Ia) or Formula (Ib) in which R₂ and R₄ are each lower alkoxycarbonyl can be prepared from the diacetyl derivatives of the compound of formula (II) above in which R′ is methyl, by oxidation with CrO₃ to form the compound of the formula (IV):

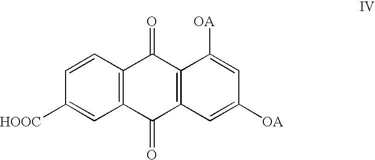

which is then dimerized by the method of Spitzner (Angew. chem. Int. Ed., 16, 46 (1977)) to form a compound of Formula (Ia) or Formula (Ib) in which R is carboxy which is then esterified with lower alkanol to obtain the desired product of Formula (Ia) or Formula (Ib) in which R₂ and R₄ are lower alkoxycarbonyl.

The compounds of Formula (Ia) or Formula (Ib) in which each R at positions 1 and 6 is alkylamino or hydroxy alkylamino may be obtained by amination of the corresponding compound of Formula (Ia) or Formula (Ib), in which each R is alkoxy, with an alkyl amine such as butyl amine, or a hydroxyalkyl amine such as ethanolamine.

The pharmaceutical compositions of the invention may be administered to the patient by standard procedures. The amount of compound to be administered and the route of administration will be determined according to the kind of tumor, stage of the disease, age and health conditions of the patient. The preferable routes of administration are systemic routes, the intravenous or the oral routes being preferred. Topical application with suitable topical compositions is also within the scope of the present invention.

The compounds of the present invention can be used to prevent and treat fibrosis or inflammatory reaction caused by radiotherapy to various types of cancers and their metastases, including, but without being limited to, breast carcinoma, lung carcinoma, squamous cell carcinoma, basal cell carcinoma, melanoma, Kaposi sarcoma, prostate carcinoma, hemangioma, meningioma, astrocytoma, neuroblastoma, carcinoma of the pancreas, gastric carcinoma, colorectal carcinoma, colon carcinoma, transitional cell carcinoma of the bladder, and carcinoma of the larynx, chronic myeloid leukemia, acute lymphocytic leukemia, acute promyelocytic leukemia, multiple myeloma, T-cell lymphoma, B-cell lymphomas hepatic cancer, gallbladder, bronchial and skin cancers. In addition, the compounds of the present invention can be used to prevent or treat inflammation caused by other types of radiation, such as laser, ultraviolet or infrared radiation, for example in microwave thermotherapy.

The compound used according to the invention can be formulated by any required method to provide pharmaceutical compositions suitable for administration to a patient.

The novel compositions contain, in addition to the active ingredient, conventional pharmaceutically acceptable carriers, diluents and the like. Solid compositions for oral administration, such as tablets, pills, capsules or the like, may be prepared by mixing the active ingredient with conventional, pharmaceutically acceptable ingredients such as corn starch, lactose, sucrose, sorbitol, talc, stearic acid, magnesium stearate, dicalcium phosphate and gums, with pharmaceutically acceptable diluents. The tablets or pills can be coated or otherwise compounded with pharmaceutically acceptable materials known in the art to provide a dosage form affording prolonged action or sustained release. Other solid compositions can be prepared as microcapsules for parenteral administration. Liquid forms may be prepared for oral administration or for injection, the term including subcutaneous, intramuscular, intravenous, and other parenteral routes of administration. The liquid compositions include aqueous solutions, with or without organic cosolvents, aqueous or oil suspensions, emulsions with edible oils, as well as similar pharmaceutical vehicles. In addition, the compositions of the present invention may be formed as encapsulated pellets or other depots, for sustained delivery.

The active dose for humans is generally in the range of from 0.1 micrograms to about 1 mg per kg body weight, in a regimen of one or more times a day. However, administration at longer intervals may also be possible, for compounds or formulations having prolonged action.

In general, the preferred range of dosage is from 1 to 200 micrograms per kg body weight. It is evident to one skilled in the art that dosage form and regimen would be determined by the attending physician, according to the disease to be treated, method of administration, and the patient's general condition. It will be appreciated that the most appropriate administration of the pharmaceutical compositions of the present invention will depend first and foremost on the clinical indication being treated. A non-limiting example of a typical treatment dosage in humans, is orally administration of hypericin with a maximal dose of 0.1 mg/kg 48 hr following exposure to ionizing irradiation.

It will be appreciated by the skilled artisan that in some instances treatments may beneficially include the administration of the compositions according to the present invention in conjunction with a depot or medical device.

The invention will now be illustrated by the following non-limiting Examples.

### EXAMPLES

All the compounds used in the present invention are known compounds and methods for their preparation are disclosed in the literature and in detail in PCT Publication WO 99/06347 the teachings of which are incorporated herein in their entirety by reference as if set forth herein.

### Example 1

### Evaluation of Cytokine Secretion Profile and Pathohistological Effect of Hypericin in Lung-Irradiated Mice

### Mice

Male BABL/c mice 6-8 weeks old mice restrained in modified Perspex tubes, anaesthetized by Ketamine & Xylazine.

The mice are divided into six groups:

i. Control

ii. Hypericin 25 μg/animal

iii. Hypericin 100 μg/animal

iv. irradiation alone

v. Hypericin 25 μg/animal following irradiation

vi. Hypericin 100 μg/animal following irradiation.

### Irradiation:

In each experiment a different dose of lung irradiation is administered. The irradiation is performed on anesthetized mice. The mice weight is evaluated at the beginning of the experiment. Six MV X-ray from a linear accelerator and a 1.5 cm bolus on the surface irradiates the whole thorax. Control mice are subjected to sham-irradiation.

### Protocol:

To analyze the expression of inflammatory cytokines, mice are irradiated with 20 Gy and sacrificed 6 hr and 24 hr after the irradiation. Tissues are taken for mRNA extraction. For histological examination, mice are being administered 10 Gy irradiation and are sacrificed after 3 and 12 weeks. Hypericin is applied intraperitoneally to treated groups 2, 12, 24, 48 hrs after irradiation and once every other day for 10 days. At the end of each experiment the mice are weighed and bled from the retro-orbital plexus. The collected sera are analyzed for pro-inflammatory cytokines (TNF-α, IL-6 and CRP) using commercially-available ELISA kits.

### Example 2

### The Effect of Hypericin on Secreted Cytokines in Irradiated Lung Cells

The effects of hypericin, and related molecules, on cytotoxicity and intracellular oxidative stress in normal lung fibroblasts and lung cancer cell line are tested in vitro. Human lung cancer A549 cells and human normal lung fibroblast WI-38, obtained from ATCC are routinely cultured in complete MEM medium and maintained in a 37° C. incubator with 5% CO₂ and 95% air. Cells are irradiated (9 Gy in a single fraction) with a 6 MeV electron beam generated by a linear accelerator at a dose rate of 300 cGy/min. After irradiation, exponentially growing cells are treated with or without hypericin at various doses from 0.1-100 μg/ml. The cells are grown in an incubator, the growth medium is collected from each group and analyzed for pro-inflammatory cytokines (TNF-α, IL-6 and CRP) using commercially-available ELISA kits. Viable cells are counted on day 2, 4, 6 and 8 after treatment.

### Example 3

### Pig Skin Studies

The pig skin model is described by J. W. Hopewell (van den Aardweg et al 1988). The structural similarities between human and pig skin underlie the relevance of the model. Although some pig skin studies involve external beam irradiation, a particularly convenient radiation source is discs containing β⁻-emitting isotopes, typically ⁹⁰Sr. The sources can be held to the skin, or for higher doses, taped to the animal, for appropriate periods of time to deliver the required dose. The discs are typically about 2 cm in diameter so that an array of different doses can be arranged on the flanks of a single animal. The “acute” reactions (3-9 weeks after irradiation) result from radiation damage to the basal cells of the epidermis, and manifest as erythema, and dry and moist desquamation. The “late” reactions (10-16 weeks) result from radiation effects to the dermal vascular connective tissue, characterized by a dusky mauve erythema and necrosis. The scoring of erythema and necrosis is well described. The tested compounds are formulated in a propylene glycol cream containing 10% DMSO. The extent and kinetics of penetration are followed by fluorescence microscopy of frozen skin sections, to enable optimization of delivery.

### Example 4

### Effects of Hypericin on the Development of Inflammatory Bowel Disease (IBD) In Vivo

The aim of the study is to evaluate the inhibitory effects of hypericin on the development of inflammatory bowel disease (IBD) in mice models.

### Mice

36 male BABL/c mice anesthetized with Ketamine & Xylazine, are divided into six groups:
Control
Dextran sulfate sodium salt (DSS) alone −8000 MW
DSS with Hypericin 25 μg/animal
DSS with Hypericin 75 μg/animal
Hypericin 25 μg/animal
Hypericin 75 μg/animal

### Protocol:

Acute IBD was generated in BALB/C mice (6 mice per group) by DSS administered via the drinking water (3.5% w/v) for 7 days. Hypericin was administered to these animals intraperitoneally at doses of 25 and 75 μg/mouse beginning 48 hrs prior to initiation of DSS administration and at 48 hr intervals thereafter. After 16 days the mice were sacrificed with high dose of sodium pentobarbital, the gastro-intestinal tract removed, its overall length measured and evaluated compared to control untreated healthy mice.

### Results:

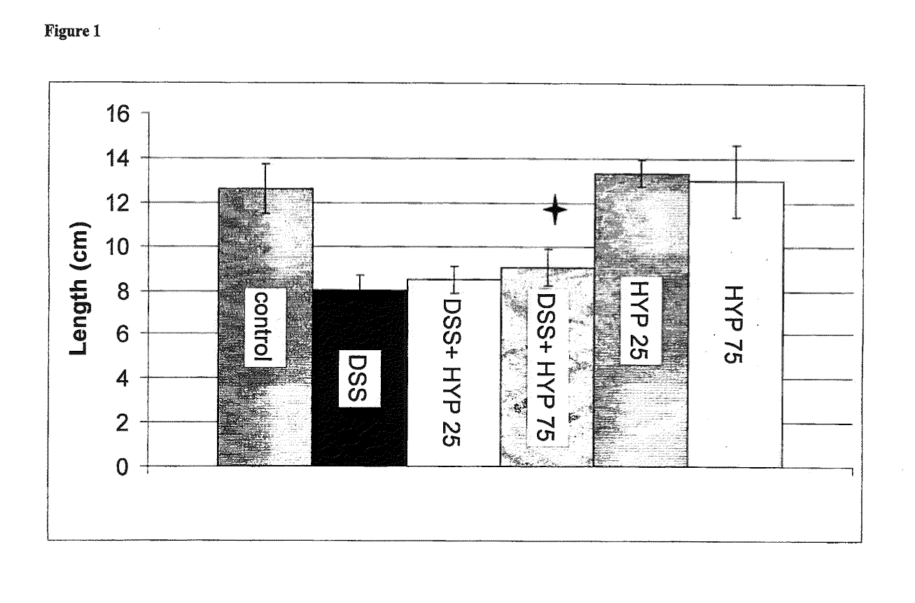

As presented in FIG. 1, 3.5% DSS (8000 MW) administration results in shortening of the colon length from 12.6 cm in the control group to 8.016 cm in the DSS treated group (P≦2.08002E-05, two tailed student's T test), while administration of Hypericin 75 μg/mouse with DSS significantly increased the colon length in comparison to DSS alone (9.067 cm; P≦0.039). Administration of Hypericin 25 or 75 μg/mouse without DSS did not alter the colon length.

Thus as clearly presented by this experiment, Hypericin administration prevents the colon shortening in mice with IBD. It has been well established in this model that the colon shortening is due to proinflammatory processes including TNFα secretion and lymphocytes infiltrations.

### Example 5

### Preventing the Cell Death Induced by TNFα Using Hypericin

The aim of the study is to evaluate the ability of hypericin to salvage non malignant cells from death induced by TNFα.

Mouse L cells (ATCC) were cultured in complete MEM medium in 37° C. incubator with 5% CO₂ and 95% humidity. The culture cells were divided to 8 separate groups:
i. untreated control
ii. Hypericin 0.5 μg/ml
iii. Hypericin 1 μg/ml
iv. TNFα10 u/ml
v. TNFα25 u/ml
vi. TNFα10 u/ml and Hypericin 0.5 μg/ml
vii. TNFα10 u/ml and Hypericin 1 μg/ml
viii. TNFα 25 u/ml and Hypericin 0.5 μg/ml
ix. TNFα25 u/ml and Hypericin 1 μg/ml.

Hypericin was applied to the cells 48 hr prior to TNFα administration. The experiment was terminated 24 hrs after TNF administration and cell viability evaluated by MTT assay.

### Study Results:

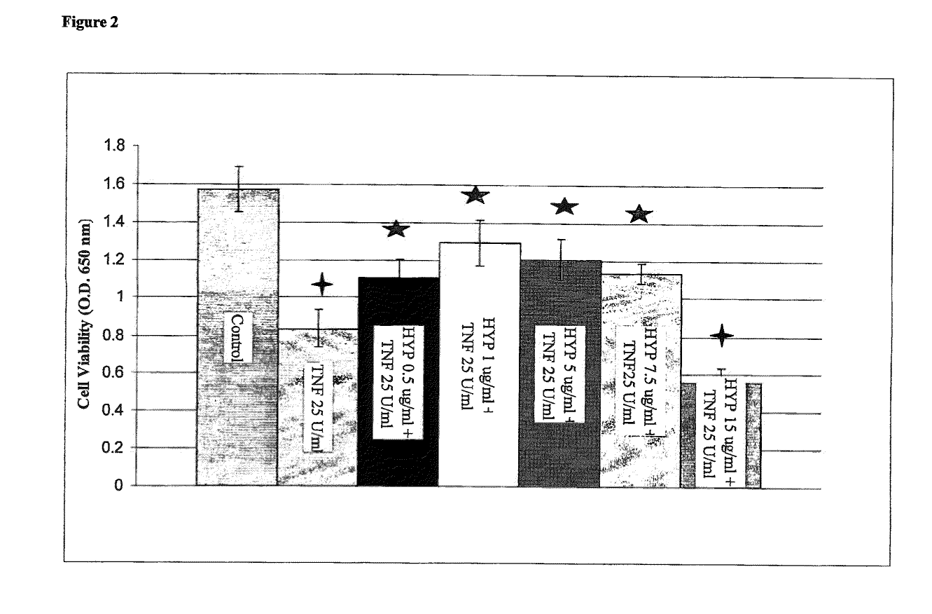

Significant levels of cell death were induced in L cells by 10 u/ml and 25 u/ml TNFα compared to the control group (♦ P≦0.000306172 and ♦ P≦2.06827E-06, respectively, two tailed student t test). Hypericin administration alone did not alter cell viability compared to the control group. However, administration of Hypericin at concentrations of 0.5 and 1 μg/ml before the administration of 10 u/ml TNFα prevented cell death significantly (★P_{(Hyp 0.5 μg/ml+TNF 10u/ml)}≦0.025758 ★ P_{(HYP 1 μg/ml+TNF 10 u/ml)}≦0.014777). Administration of Hypericin at concentrations of 0.5 and 1 μg/ml before the administration of TNFα25 u/ml also prevented cell death significantly (∈ P_{(Hyp 0.5 μg/ml+TNF 25u/ml)}≦0.023446 ★ P_{(Hyp 1 μg/ml+TNF 25u/ml)}≦0.047198) (Shown in FIG. 2).

Thus it is concluded that Hypericin prevents TNFα induced cell death in normal cells.

### Example 6

### Preventing the Cell Death Induced by TNFα Using Hypericin—Evaluation with the Hemacolor Assay (Keisari v. 1992)

The aim of the study is to evaluate the ability of hypericin to salvage non malignant cells from death induced by TNFα using an alternative method of quantification—Hemacolor assay.

Mouse L cells (ATCC) were cultured in complete MEM medium in 37° C. incubator with 5% CO₂ and 95% humidity. The culture cells were divided to 6 separate groups:
i. untreated control
ii. TNFα25 u/ml
iii. TNFα25 u/ml and Hypericin 0.5 μg/ml
iv. TNFα25 u/ml and Hypericin 1 μg/ml
v. TNFα25 u/ml and Hypericin 5 μg/ml
vi. TNFα 25 u/ml and Hypericin 7.5 μg/ml.

Hypericin was applied to the cells 48 hr prior to TNFα administration. The experiment was terminated 24 hrs after TNF administration and cell viability evaluated using the Hemacolor assay.

### Study Results:

Significant levels of cell death were induced in L cells by 25 u/ml TNFα compared to the control group ( [symbol] P≦0.0262, two tailed student t test). Administration of Hypericin at concentrations of 0.5, 1, 5 and 7.5 μg/ml before the administration of 25 u/ml TNFα prevented cell death significantly (∈ P_{(Hyp 0.5 μg/ml+TNF 25 u/ml)}≦0.0045, ★ P_{(Hyp 1 g/ml+TNF 25u/ml)}≦0.00049, ★ P_{(Hyp 5 μg/ml+TNF 25 u/ml)}≦0.00105 and ★ P_{(Hyp 7.5 ug/ml+TNF 25u/ml)}≦0.00159). Administration of Hypericin at concentrations of 15 μg/ml before the administration of TNFα25 u/ml did not prevent cell death (Shown in FIG. 2b). Thus it is concluded that Hypericin prevents TNFα induced cell death in normal cells.

### Example 7

### Effects of Hypericin on the Development of Inflammatory Skin Reactions Induced by Herpes Simplex Type 1 Virus in Guinea Pig Dorsa

The aim of the study is to evaluate the inhibitory effects of hypericin on the development of inflammatory erythema and edema following infection with herpes virus.

### Animals:

Male guinea pigs anesthetized by Ketamine & Xylazine.

Hypericin was prepared as a topical formulation in soft Vaseline (1.7 μg Hypericin/gr Vaseline, 1.2 μg Hypericin/gr Vaseline)

The animals were divided into 3 groups
Herpes virus inoculated (4)
Herpes virus and 1.7 μg Hypericin/gr Vaseline (4)
Herpes virus and 1.2 μg Hypericin/gr Vaseline (4)

### Protocol:

The guinea pigs were anesthetized with Ketamine 100 mg/ml and Xylazine 20 mg/ml (7:3), total volume of 0.5 ml/kg. Six small 4 mm crossed incisions were made in the skin. Herpes simplex type 1 virus at a titer of 10⁶ TCID/ml (Tissue culture infective dose) was applied to 4 of the 6 incisions. The two others served as controls for incision-induced mechanical inflammation in the absence of virus (controls, not infected with a virus). Hypericin was applied topically 3× per day for 3 consecutive days and the animals evaluated for inflammation related symptoms after 96 hrs.

### Results:

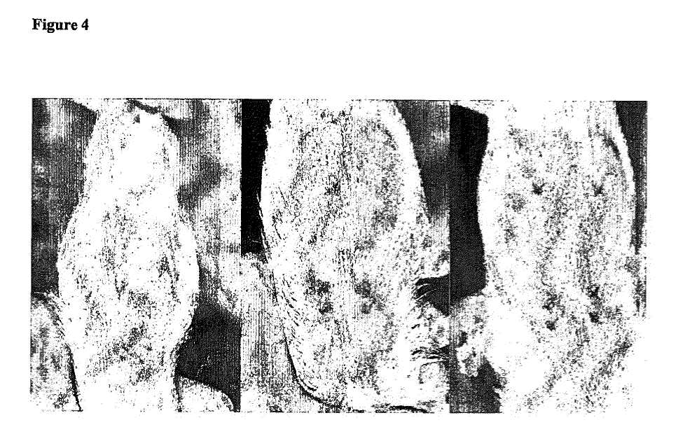

FIG. 4 depicts three animals. A control animal which was only inoculated with the virus (four lower tissue scrapings) but received no hypericin is shown on the left. An animal which was inoculated with the virus and administered with 1.2 μg Hypericin/gr is shown in the middle and a third animal which inoculated with the virus and received 1.7 μg Hypericin/gr is shown on the right. The results show that the inflammatory erythema which follows infection with herpes viruses can be significantly inhibited by treatment of the animals with hypericin.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

Andreyev J, 2005 Gastrointestinal complications of pelvic radiotherapy: are they of any importance?, Gut, 54:1051-1054.
Abratt et al. (2004) Pulmonary complications of radiation therapy, Clin. Chest Med., 25), 167-177.
Buchi K. (1991) Radiation proctitis: therapy and prognosis. JAMA 265, 1180.
Carroccio et al (2006) Recenti Prog Med. 97:108-12.
Chen et al. (2005) Caffeic acid phenthyl ester decreases acute pneumonitis after irradiation in vitro and in vivo. BMC Cancer 5, 158.
Chen et al. (2006) A systematic review of the effectiveness of adalimumab, etanercept and infliximab for the treatment of rheumatoid arthritis in adults and an economic evaluation of their cost-effectiveness. Health Technol Assess. 10(42):iii-iv, xi-xiii, 1-229.
Elliot, M., et al., (1993) Arthritis and Rheum., 36:1681.
Fiers W., (1991) FEBS Letters, 285, p. 199.
Hallahan D. E., Sprigs D. R., Beckett M. A. et al. (1989) Increased tumor necrosis factor alpha mRNA after cellular exposure to ionizing radiation. Proc. Natl. Acad. Sci. (USA) 86, 10 104-10107.
Hallahan D. E. & Virudachalam S. (1997) Ionizing radiation mediates expression of cell adhesion molecules in distinct histological patterns within the lung. Cancer. Res. 57, 2096-2099.
Hong et al., (1999) Rapid induction of cytokine gene expression in the lung after single and fractionated doses of radiation. Int Radiat Biol., 75, 1421-1427.
Keisari Y., (1992) A colorimetric microtiter assay for the quantitation of cytokine activity on adherent cells in tissue culture. J Immunol Methods., 146(2), 155-61.
Kim J., J. of Immunology, 2002, 169:1535-1541.
Kolls, J., et al., (1994)) Proc. Natl. Acad. Sci. USA, 91:215.
Krivenko S et al. (1992) Ionizing radiation increases TNF/cachectin production by human peripheral blood mononuclear cells in vitro. Int. J. Hematol. 55, 127-130.
Johnston et al. (1996) Early and persistent alterations in the expression of interleukin-1 alpha, interleukin-1 beta, and tumor necrosis factor alpha mRNA levels in fibrosis-resistant and sensitive mice after thoracic irradiation. Radiat. Res. 145, 762-767.
Lavie et al., (1989) Studies of the mechanisms of action of the antiretroviral agents hypericin and pseudohypericin. Proc. Nat. Acad. Sci. (USA) 86:5963.
Lavie et al., (1994) The chemical and biological properties of hypericin—A compound with a broad spectrum of biological activities. Medicinal Res. Rev. 15: 111-119.
Meruelo et al., (1988) Therapeutic agents with dramatic antiretroviral activity and little toxicity at effective doses: aromatic polycyclic diones hypericin and pseudohypericin. Proc. Nat. Acad. Sci. (USA) 85: 5230-5324.
Miyazali et al., (1995) Expression of a tumor necrosis factor-a transgene in murine lung causes lymphocytic and fibrosing alveolitis. J. Clin. Invest. 96, 250-259.
Moore et al., (2000) Rectal bleeding after radiation therapy for prostate cancer: endoscopic evaluation. Radiology. 217, 215-8.
Morgan G. W. & Breit S. N. (1995) Radiation and the lung: a reevaluation of the mechanisms mediating pulmonary injury. Int. J. Radiat. Oncol. Biol. Phys. 31, 361-369.
Paludan S R et al., Virus-cell interactions regulating induction of tumor necrosis factor alpha production in macrophages infected with herpes simplex virus. J. Virol. 2001; 75(21):10170-8.
Perry A and Schmidt R E., (2006) Cancer therapy-associated CNS neuropathology: an update and review of the literature. Acta Neuropathol (Berl). February 4, 1-16.
Rubin et al., (1995) A perpetual cascade of cytokines postirradiation leads to pulmonary fibrosis. Int. J. Radiat. Oncol. Biol. Phys. 33, 99-109.
Shankar G. & Cohen D. A. Idiopathic pneumonia syndrome after bone marrow transplantation: the role of pre-transplant radiation conditioning and local cytokine dysregulation in promoting lung inflammation and fibrosis International Journal of Experimental Pathology, 82, 101-113.
Sheline G E (1977) Radiation therapy of brain tumors. Cancer 39, 873-881.
Sime et al., (1998) Transfer of tumor necrosis factor-alpha to rat lung induces severe pulmonary inflammation and patchy interstitial fibrogenesis with induction of transforming growth factor-beta1 and myofibroblasts. Am. J. Pathol. 153, 825-832.
Stone et al. (2003) Effects of radiation on normal tissue: consequences and mechanisms. Lancet Oncol 2003; 4: 529-36.
Talkahashi et al., (1989) Hypericin and pseudohypericin specifically inhibit protein kinase C: possible relation to their antiretroviral activity. Biochem. Biophys. Res. Commun 165:1207.
Tang et al., (1990) Virucidal activity of hypericin against enveloped and non-enveloped DNA and RNA viruses. Antiviral Res. 13:313-326.
Tartakovsky et al. (1993) Low doses of radiation induce systemic production of cytokines: possible contribution to leukemogenesis. Int. J. Cancer 55, 269-274.
Thornton et al. (1996) Both in vitro and in vivo irradiation are associated with induction of macrophage-derived fibroblast growth factors. Clin. Exp. Immunol. 103, 67-73.
Thrall et al., (1997) Role of tumor necrosis factor-alpha in the spontaneous development of pulmonary fibrosis in viable motheaten mutant mice. Am. J. Pathol. 151, 1303±1310.
Tracy, K. J. and Cerami, A., (1993) Ann. Rev. Cell. Biol., 9:317.
Vowels B R, Induction of proinflammatory cytokines by a soluble factor of Propionibacterium acnes: implication for chronic inflammatory acne. Infect. Immun. 63:3158, Basal E. J. Microbiol. 2004; 42(2):117-25.
Zhang et al. (Clinical Cancer Research, 2, 843, 1996.

1-42. (canceled)

43. A method for preventing or inhibiting a TNFα-mediated inflammatory condition, the method comprising administering to a patient in need thereof a therapeutically effective amount of a compound of the general formula (Ia) or (Ib): 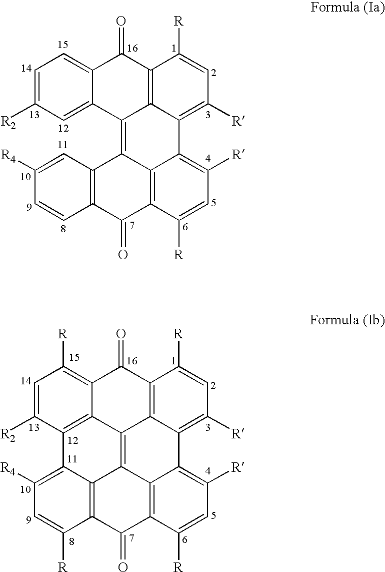
wherein R is selected from the group consisting of hydroxy, C₁-C₁₀ alkoxy, NH—C₁-C₁₀ alkyl, and NH-hydroxy(C₁-C₁₀)alkyl; R′ is selected from the group consisting of hydroxy and C₁-C₁₀ alkoxy; and R₂ and R₄ are independently selected from the group consisting of hydrogen, hydroxy, chloro, bromo, C₁-C₁₀ alkyl, C₁-C₁₀ alkoxy, and C₁-C₁₀ alkoxycarbonyl; thereby preventing or inhibiting a TNFα-mediated inflammatory condition.

44. The method of claim 43, wherein said compound of formula Ia is hypericin.

45. The method of claim 43, wherein said compound of formula Ib is selected from the group consisting of:
1,3,4,6-tetrahydroxyhelianthrone;
1,3,4,6-tetramethoxyhelianthrone;
10,13-dimethyl-1,3,4,6-tetramethoxyhelianthrone;
10,13-dimethyl-1,3,4,6-tetrahydroxyhelianthrone;
10,13-di(methoxycarbonyl)-1,3,4,6-tetramethoxyhelianthrone;
1,6-di-N-butylamino-3,4-dimethoxy-helianthrone;
1,6-di-N-butylamino-3,4-dimethoxy-10,13-dimethyl-helianthrone; and
1,6-di-(N-hydroxyethylamino)-3,4-dimethoxy-helianthrone.

46. The method of claim 43, wherein the condition is selected from the group consisting of: inflammatory bowel disease, ulcerative, acute or ischemic colitis, Crohn's disease and cachexia (wasting syndrome).

47. The method of claim 43, wherein the condition is septic shock (sepsis, endotoxic shock) or disseminated bacteremia.

48. The method of claim 43, wherein the condition is a neurodegenerative disease.

49. The method of claim 48, wherein the neurodegenerative disease is selected from the group consisting of: Alzheimer's disease, neurological lesions associated with diabetic neuropathy, demyelinating disorders other than autoimmune demyelinating disorders, retinal degeneration, glaucoma and peripheral neuropathies.

50. The method of claim 43, wherein the condition is glaucoma.

51. The method of claim 43, wherein the condition is a bacterially or virally induced inflammation.

52. The method of claim 51, wherein the virally induced inflammation is caused by a virus selected from the group of: herpes simplex virus type 1, herpes simplex virus type 2, varicella zoster virus and varicella virus.

53. The method of claim 51, wherein the bacterially induced inflammation is caused by a bacteria selected from the group consisting of: Streptococcus, Meningococcus, Staphylococcus bacteria and Propionibacterium acnes.

54. A method for preventing or inhibiting a cytokine mediated inflammation, fibrosis or vasculopathy caused by exposure to radiation, the method comprising administering to a patient in need thereof a therapeutically effective amount of a compound of the general formula (Ia) or (Ib): 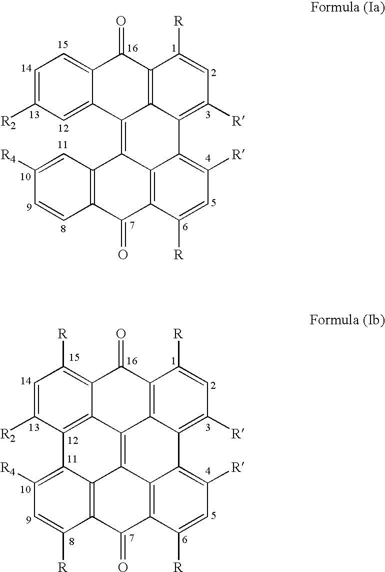
wherein R is selected from the group consisting of hydroxy, C₁-C₁₀ alkoxy, NH—C₁-C₁₀ alkyl, and NH-hydroxy(C₁-C₁₀)alkyl; R′ is selected from the group consisting of hydroxy and C₁-C₁₀ alkoxy; and R₂ and R₄ are independently selected from the group consisting of hydrogen, hydroxy, chloro, bromo, C₁-C₁₀ alkyl, C₁-C₁₀ alkoxy, and C₁-C₁₀ alkoxycarbonyl; thereby preventing or inhibiting a cytokine mediated inflammation, fibrosis or vasculopathy caused by exposure to radiation.

55. The method of claim 54, wherein said compound of formula Ia is hypericin.

56. The method of claim 54, wherein said compound of formula Ib is selected from the group consisting of:
1,3,4,6-tetrahydroxyhelianthrone;
1,3,4,6-tetramethoxyhelianthrone;
10,13-dimethyl-1,3,4,6-tetramethoxyhelianthrone;
10,13-dimethyl-1,3,4,6-tetrahydroxyhelianthrone;
10,13-di(methoxycarbonyl)-1,3,4,6-tetramethoxyhelianthrone;
1,6-di-N-butylamino-3,4-dimethoxy-helianthrone;
1,6-di-N-butylamino-3,4-dimethoxy-10,13-dimethyl-helianthrone; and
1,6-di-(N-hydroxyethylamino)-3,4-dimethoxy-helianthrone.

57. The method of claim 54, wherein the radiation is selected from the group consisting of: ionizing radiation, laser irradiation, microwave irradiation, ultraviolet irradiation, ultrasonic thermotherapy and infrared irradiation.

58. The method of claim 54 wherein the compound is administered following the exposure to radiation.

59. The method of claim 58 wherein the compound is administered immediately following to the exposure to radiation.

60. A method for protecting biological material of a subject from irradiation damage, the method comprising administering to a subject in need thereof a compound of formula (Ia) or (Ib); 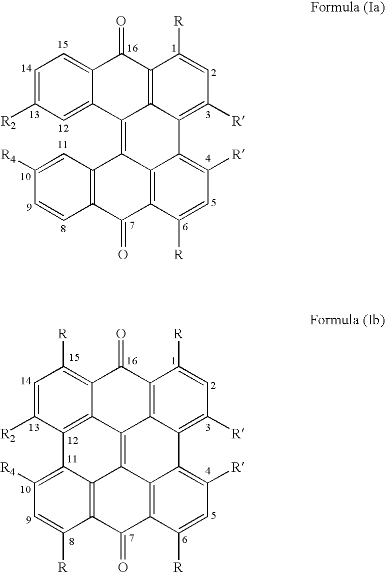
in an amount effective to protect biological material of the subject from irradiation damage; wherein R is selected from the group consisting of hydroxy, C₁-C₁₀ alkoxy, NH—C₁-C₁₀ alkyl, and NH-hydroxy(C₁-C₁₀)alkyl; R′ is selected from the group consisting of hydroxy and C₁-C₁₀ alkoxy; and R₂ and R₄ are independently selected from the group consisting of hydrogen, hydroxy, chloro, bromo, C₁-C₁₀ alkyl, C₁-C₁₀ alkoxy, and C₁-C₁₀ alkoxycarbonyl; thereby protecting biological material of the subject from irradiation damage.

61. The method of claim 60, wherein said compound of formula Ia is hypericin.

62. The method of claim 60, wherein said compound of formula Ib is selected from the group consisting of:
1,3,4,6-tetrahydroxyhelianthrone;
1,3,4,6-tetramethoxyhelianthrone;
10,13-dimethyl-1,3,4,6-tetramethoxyhelianthrone;
10,13-dimethyl-1,3,4,6-tetrahydroxyhelianthrone;
10,13-di(methoxycarbonyl)-1,3,4,6-tetramethoxyhelianthrone;
1,6-di-N-butylamino-3,4-dimethoxy-helianthrone;
1,6-di-N-butylamino-3,4-dimethoxy-10,13-dimethyl-helianthrone; and
1,6-di-(N-hydroxyethylamino)-3,4-dimethoxy-helianthrone.

63. The method of claim 60 wherein the irradiation damage is caused by cancer radiotherapy.

64. The method of claim 60 wherein the irradiation is selected from the group consisting of: ionizing radiation, laser irradiation, microwave irradiation, ultraviolet irradiation, ultrasonic thermotherapy and infrared irradiation.

65. The method of claim 60 wherein the damage is associated with intraocular inflammation caused by laser irradiation.

66. The method of claim 60 wherein the compound of formula (Ia) or (Ib) is administered following the exposure to irradiation.

67. The method of claim 66 wherein the compound of formula (Ia) or (Ib) is administered immediately following completion of the exposure to irradiation.

68. The method of claim 65, wherein the administering comprises administering an effective amount of a compound of formula (Ia) or (Ib) or an acid addition salt thereof, to a local site of the eye.
